(19) United States
(12) Patent Application Publication
(10) Pub. No.: US 2011/0070632 A1
(43) Pub. Date: Mar. 24, 2011

(54) Photo bioreactor and cultivation system for improved productivity of photoautotrophic cell cultures

(75) Inventors: Anand Swarup Katoch, Rockville, MD (US); Rakhi Anand Katoch, Rockville, MD (US)

(73) Assignee: BioCetane Inc., Wasaga Beach (CA)

(21) Appl. No.: 12/586,218

(22) Filed: Sep. 18, 2009

(51) Int. Cl. C12M 1/36; C12M 1/00; B08B 5/00

(52) U.S. Cl. 435/286.6; 435/292.1; 15/405; 15/4

(57) ABSTRACT

This invention relates to open or close top photobioreactor apparatus for improved productivity with a small footprint, that can be placed in varied temperature zones, above or below ground on land or in water or use in space-station for conducting aqueous aerobic or anaerobic; continues or batch wise cultivation and harvesting of photoautotrophic organism's.

More particularly this invention concerns with a novel means and process for uniform optical dispersion and optical enhancement by utilizing a solar energy collection unit attached to a photo-emitting system which emits full spectrum or specific wavelength light substantially uniformly and radially along its length.

### TECHNICAL FIELD OF THE INVENTION

The present invention relates to improved productivity photobioreactor apparatus and cultivation system for growing and harvesting algae or other photoautotrophic organisms in Photo-Bioreactor or substantially sealed environment. More particularly, the invention is concerned with high efficient commercial or lab-scale Photo-Bioreactors and Photo-Bioreactor systems for uniform optical dispersion and optical enhancement of photosynthetic productivity of algae or other photoautotrophic organisms in a small footprint. Certain embodiments concern methods, compositions, apparatus and a system for production of useful products from algae, such as bio-fuels (e.g. bio-diesel, methanol, ethanol), bio-polymers, chemical precursors and/or animal or human food. Other embodiments concern use of such a system to remove carbon dioxide from sources such as power plant emissions or heavy metals from water.

### BACKGROUND OF THE INVENTION

This invention relates to a method for efficiently growing photoautotrophic microorganisms in liquid suspension, and specifically to mass culture unicellular algae. In the past decade, there has been considerable activity relating to production of photoautotrophic micro-algae for commercial purposes. Special industries aiming to produce health food, food additives, animal feed, bio-fertilizers and an assortment of natural products (most notably β-carotene) have been established. Recently, micro-algae have been suggested as a means to sequester carbon from the industry, and hydrogen producing algae as a source of energy.

Historically most efforts have been invested in developing the optimum nutrients for any specific algae. This includes means to saturate the photosynthetic system with carbon dioxide. Various methods and equipment have been employed for the artificial culturing of photoautotrophic organism especially micro-algae. The first generation of photobioreactor is based on the use of shallow open pond (15-100 cm) exposed to sunlight and agitated with paddle wheel. Such ponds are proved to require a large footprint, laborious to clean and have poor productivity, due to little or no provision in controlling the seasonal and daily climatic variation such as degree of exposure to light, temperature and respiration leading to frequent catastrophic loss of cultures. This open raceway is also impractible for production of pure pharmaceutical grade or food grade products as they are subject to contamination by dust, other microorganism, insect and environmental pollutants. In low temperature zone countries such as Canada, Russia and China, outdoor operation in the autumn and winter seasons are out of synchrony with the light and temperature regimes.

A more sophisticated approach has involved growing algae cultures in plastic-covered trenches and ponds, optionally having electricity powered pumps and agitators. These configurations reduce the chances of contamination of the culture and permit more accurate control of temperature, respiration and other parameters. Such configurations are still quite inefficient in terms of providing adequate and uniform amount of light to the algal cells particularly when sunlight is the sole source of light.

The second generation of photobioreactor involved cultivation under more closed cultivation system, as they can overcome the majority of limitations allotted to the conventional shallow lagoons. The most popular closed cultivation system are the tubular photobioreactor such as U.S. Pat. Nos. 5,137,828; 5,242,827 and 6,174,720 whose configuration allows to reach high production rates due to the optimization of their light path and automated control of parameter such as pH, nutrients, temperature and carbon dioxide. The drawback of these systems is a need of large footprint, too expensive, difficult to clean, high drain down time and high utility cost.

Third generation of photobioreactor U.S. Pat. Nos. 5,104,803; and 5,162,051 were developed to eliminate the effect of photoperiod due to season, localization and diurnal cycle, by use of artificial light as an energy source for the growth of photoautotrophic organisms. As these systems had high utility cost due to artificial light, maintenance difficulty and also being too expensive to scale up, they rarely exceeded the stage of prototype or lab scale photobioreactors.

A primary design factor for modern photobioreactor involve providing a means for uniformly exposing the cell in the algal culture to the optimum amount of visible light. Efficient photobioreactor design requires that light be provided at the required intensities, duration and wavelength based on pigments present in the micro algae. Like many plants, algae are quite sensitive to the amount and kind of light. Different pigments absorb/harvest different regions of visible light energy in the light wavelength range of 400-500 nm and 600-700 nm. Excessive light intensity can damage algal collection antenna causing photo-inhibition and photo-oxidation and eventually kill algae cells. Too little light results in low level of photosynthesis. This suggests that individual algae culture have to be considered by important pigments identification for light harvesting as it dictate the design of the lighting system for a photobioreactor.

Still further development U.S. Pat. No. 5,614,378 have recently been made by growing algae in tubes in which fiber optic light guides are inserted in the tubes. These fibers uniformly diffuse a large proportion of the light passing through them. A solar concentrator is used to concentrate solar light onto one end of the fibers. The fiber optics in this arrangement occupies a large proportion of the reactors volume and a large proportion of the light is lost through the end of the fibers.

Like conventional fermentation process, for industrial applications it is usually desirable to use a high cell density in large scale batches to result in economics of scale. Many of the same consideration apply to autotrophic culture as to heterotrophic culture. In addition to light intensity and specific wavelength, one must take into account the competition for nutrients, respiratory demands, viscosity and pumpability of the culture medium. The principle hurdle in scale up of photobioreactor to achieve a viable commercial scale production is light limitation, both in terms of light delivery and distribution and energy expenditure. The current methods of mass cultivation of marine micro algae under artificial lighting or natural illuminations in green house at an algal density for example of 10 g/L concentration would yield an 86% loss of light energy at 1 mm depth Ogbonna and Tanaka (1997) leaving a significant percentage of the cells in complete darkness at the given time. Moreover, when lighting is provided by artificial lamps (such as fluorescent, high pressure sodium or incandescent) in close proximity to the bioreactor vessel, the comparatively poor luminous efficacy and dissipation of heat from the lamp present a constant problem.

It is also observed that penetration power of sunlight is low in algal growth medium and majority of sunlight is seen to reflect back in uncontrolled open raceway systems leaving the algae to starve for light. Therein lays the primary motivation for using filtered sunlight for lighting purposes in photobioreactors. The only effective way of increasing cell densities while maintaining a uniform amount of light is to employ a relative short path length or continuously keep the algae in motion.

A significant need to create large scale photobioreactor system capable of efficiently using solar energy by integrating solar technologies into multi-use hybrid systems that better utilize the entire solar energy spectrum served as partial precursor to this invention.

### SUMMARY OF THE INVENTION

It is therefore a broad objective of the present invention to ameliorate the disadvantages of the above described known devices, and to provide a bioreactor and process which permits a substantially gain in the net biomass, without being substantially more complex to operate then system heretofore used. The mass culture photobioreactor disclosed can precisely regulate many variables so that the cells harvested can be controlled to be of chosen chemical compositions and produced at rates representing high and nearly constant conversion efficiencies of sunlight into stored chemical free energy. In this way, the algal product can be chosen to meet a variety of needs.

It is further object of the present invention to provide a photobioreactor in which the average volume shall be increased 7-38 times that of an average raceway volume per square feet footprint, and the average light intensity penetration is increased.

Still further objective of the present invention is to provide a photobioreactor in which the spectral quality of the impinging light can be adjusted by use of light filtering fluid in accordance with specific wavelength needs of the specific algae species. The support guides or channels for the fiber-optics and electro luminescence strings can be preferably cylindrical or flat, a person skilled in the art will understand that the invention is not limited to these design but dependent upon the light need of the photoautotrophic organism, size of the photobioreactor and the location. A filtering means to absorb the infrared and ultraviolet wavelengths of sunlight passing through the light guide can be installed to provide photosynthetically active wavelengths that stimulate growth and productivity for the specified algae.

Yet still further objective of the present invention is to provide a bioreactor in which temperature prevailing inside the reactor can be adjusted and controlled by use of geothermal and solar systems. The heat energy collected by the solar collector can also be used to control the temperature of algae culture medium or can be converted to electrical energy for driving pump motor and other essential system components.

Depending upon the nature of the algae the invention also provides a closed system to continuously grow and harvest algae from the medium without stopping the process by using settle chamber near discharge end, a person skilled in the art will understand that the invention is not limited to belt filter harvester, skim harvester, drum filter, chamber filter press, separators or chemically using flocculants.

An additional object is to provide a closed loop air circulation system to enable the use of reactant gasses and chemicals to produce specialty products.

The invention also provides a mechanical means of cleaning the light guides or channels without causing a shutdown of the process. This eliminates a major problems observed in closed photobioreactor system leading to loss in light dispersion.

A more complete appreciation of the invention and many of the attendant advantages becomes better understood by reference to an integrated system, wherein combustion gases are treated with a photobioreactor system to mitigate pollutants and to produce large quantity of commercially useful algal biomass for food, cosmetic, polymer, medicine, automobile fuel, electricity generation etc.

Such a system can potentially be advantageously utilized for treating gases emitted by facilities such as fossil fuel e.g. coal, oil, and natural gas-fired power plants, industrial incineration facilities, industrial furnaces and heaters, internal combustion engines, etc. Integrated gas treatment/biomass-producing system can, in certain embodiments, substantially reduce the overall fossil fuel requirements of a combustion facility, while, at the same time, substantially reducing the amount of carbon dioxide and/or nitrous oxide released as an environmental pollutant. Hence provide a biotechnology-based air pollution control and renewable energy solution to fossil fuel burning facilities, such as power generating facilities

As described above, algae or other photoautotrophic organisms contained within the photobioreactors can utilize the carbon dioxide of the flue gas stream for growth and reproduction thereby producing biomass. As described above, in order to maintain optimal levels of algae or other photoautotrophic organisms within the photobioreactors, periodically biomass, is removed from the photobioreactors using either the continues belt extraction unit or by other methods of flocculation, surface tension reduction, skimming, froth separation, triple media filter or other mechanical means. This recovered wet algae slurry shall be either treated directly or dried prior to extraction of desired product or used as dried algae biomass.

In certain embodiments, the photobioreactor system has such a small foot print that it can act as a stand alone hydrogen generating and pumping system for industrial and automobile application. Where the generated hydrogen shall be scrubbed through purifier system, compressed and stored in tanks.

In addition, in certain embodiments, the photobioreactor can perform as an oxygen generator for institutions like hospital or used in space stations as a carbon dioxide scrubber, food source and oxygen source for astronauts.

### BRIEF DESCRIPTION OF DRAWINGS

FIGS. 1a, b: Is a cross-section side view of a first preferred embodiment of an underground closed-top photo-bioreactor system with solar trough.

FIGS. 2a, b: Is a cross-section side view of an alternate embodiment of the first preferred underground photo-bioreactor system, having plurality of circular hybrid solar collector system

FIGS. 3a, b: Is a cross-section side view of an alternate embodiment of an open top underground algal raceway system with hybrid solar trough

FIGS. 4a, b: Is a close up image of fiber optic light guide and cleaning system.

FIGS. 5a, b: Illustrates off the shelf hybrid solar collector

FIG. 6a: Shows a longitudinal cross section view of “above ground” sealed photo-bioreactor system with tubular hybrid optic light guide

FIG. 6b: Shows a plan view of FIG. 6(a) embodiment showing four hybrid optic tubular guides

FIG. 7a: In an alternate embodiment of “above ground” sealed photo-bioreactor with flat hybrid light guides

FIG. 7b: Shows a plan view of FIG. 7(a) embodiment showing four hybrid optic flat guides

FIGS. 8a, b: Shows a cross sectional view of an open top floating photo-bioreactor system in ocean surface with hybrid solar collector

FIGS. 9a, b: Is a cross sectional view of an alternate embodiment of a linier or flat hybrid fiber optic channel.

### DESCRIPTION OF THE PREFERRED EMBODIMENT

The present invention relates to methods of utilizing a solar energy collection unit encompassing a variety of technologies such as solar collector, solar trough, thermal collector, fiber optical collector, photo filters, geothermal temperature control system, filtration and sedimentation systems.

The “photo-bioreactor” used herein refers to an apparatus containing, or configured to contain, a liquid medium carrying at least one species of photoautotrophic organism and having partial exposure (according to desired photoperiod) to light source of a desired intensity or wavelength capable of conducting photosynthesis. The terms “photoautotrophic organism” or “biomass” used herein includes those organisms capable of photosynthetic growth, such as plant cells and unicellular or multi-cellular microorganisms (including Bacteria, Algae, Fungus). The term “biofuel” used herein includes any fuel or fuel-intermediate that derives from biomass produced in the photo-bioreactor.

A person skilled in the art will understand that the invention is not limited to the size of the photobioreactor and the depth of the system shall be dictated by the change in photo-collection technology and the needs of the photoautotrophic organism. In present time the technology provides a good side lighting and end lighting dispersion of 10000 lumens to a depth of 25 ft.

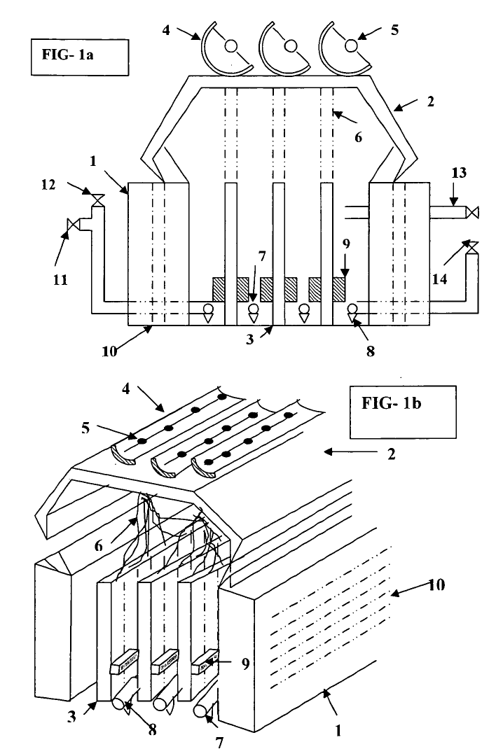

FIGS. 1a, b: Shows a cross-section side view of a first preferred embodiment of an underground closed-top (2) photobioreactor system. The photobioreactor comprise container that has a pair of support sidewalls (1) that can be formed utilizing the local material at worksite such as rock, mud and various densities of earthen clay or cement, metal, polymer or other similar durable hydrophobic non corrosive material. Since these systems can be installed in a variety of locations preferable, worksite is non-arable, thereby saving arable land for agriculture, while effectively utilizing heretofore unused land.

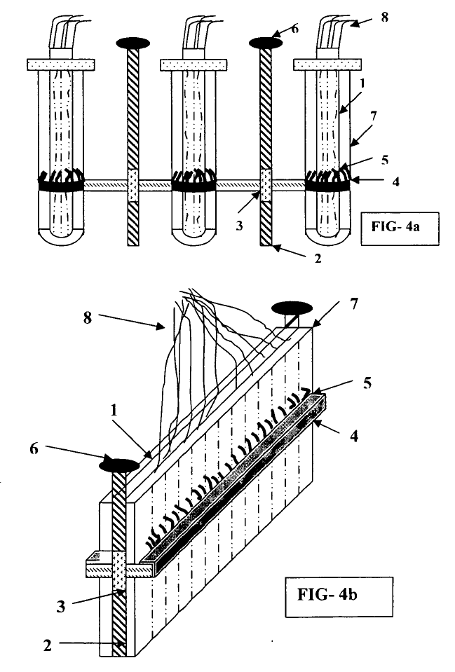

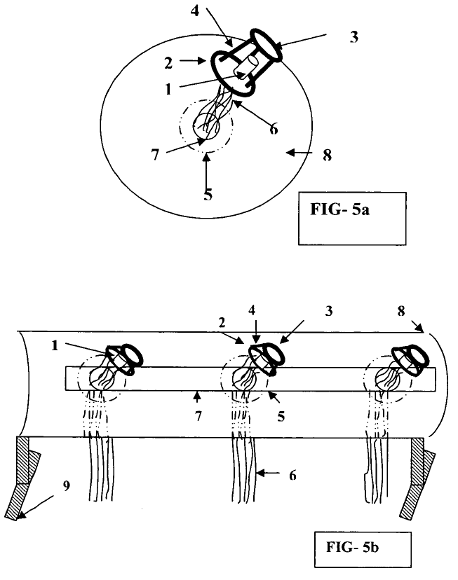

The photobioreactor system comprises of plurality of solar trough (4) with multiple photo-collectors (5) and fiber-optics (6) hybrid light guides (3) (details mentioned in FIG. 4b) linked to the solar trough (4). A person skilled in the art shall understand that the number of trough and light guide and its length can vary according to the size of the photobioreactor and for simplicity of the art shows only three solar trough and light guides. The solar trough as indicated in FIG. 5b has a grove (7) that helps to change the number of photo collectors per trough or to adjust the distance between light guides to deliver adequate light intensity as per needs of the photoautotrophic organism. The light guides (3) can be in the shape of flat curtains or separators or tubular structure with varying thickness depending upon the light needs of the photoautotrophic organism and cellular density. Solar energy collection units are arranged on a sun facing surface that is exposed to a sufficient duration of sunlight for daylight and solar energy collection.

Solar energy collection units in this exemplary illustration may be photovoltaic cells, thermal collectors, photo-collectors and the like. Consequently, soar energy system may include other supporting equipment, for example, wiring, charge collectors, steam generators, heat collectors, batteries, inverters, and the like, not shown herein for simplicity of illustration. In addition only three solar-troughs per photobioreactor are shown for simplicity of illustration. It should be understood that the quantity of solar trough and size of photobioreactor can be scaled to accommodate the needs of optimum productivity of the desired photoautotrophic organism.

Optimum amount of light intensity and duration can be controlled by the number of light guide (3) or the number of fiber optics or electro luminescence ropes per light guide or the distance between each light guide or by flashing alternate light guides.

In this exemplary embodiment, the temperature control of the photobioreactor is controlled by the help of one or more spiral geothermal heat exchange tubes (10) made up of heat transferring material, forming a sealed jacket to the enclosed system.

In a preferred embodiment, the methodology of the present invention entails providing fluid supply channel (13) and/or fluid release channel (14) to the enclosed photobioreactor. The fluid release channel opens into the sedimentation or flocculation tank that has closed loop filtration system, a person skilled in art will understand that the invention is not limited to belt harvester, drum filter, chambers filter, froth separator, mat filter etc. The biomass is harvested at the location and the clear fluid coming out of the fluid outlet is blended with nutrients and filtered before it is sent back to the photobioreactor through the fluid supply channel (13).

In the exemplary, configuration, each of the horizontally arranged injector sparger gas contacting system having parallel pipes (7) connected to sparger facing nozzle down (8); and carbon dioxide air ratio in medium is controlled by use of pneumatic valves (11, 12). This air lift system also helps the algae to be suspended and in motion.

The photo-bioreactor further comprises at least one cleaning adaptor (9) details are mentioned in FIG. 4b. A person skilled in art shall understand that the invention is not limited to flat shape of the adaptor and the choice of material for the adaptor and bristles or nozzles shall depend upon the process conditions.

Additionally the system can also have stirrer, paddle wheel, wave maker or other mechanical stirring assembly attached, to fulfill the needs of photoautotrophic organism light requirement by bringing them in close contact to the light guides. These equipments are known technology and not shown for simplicity of illustration.

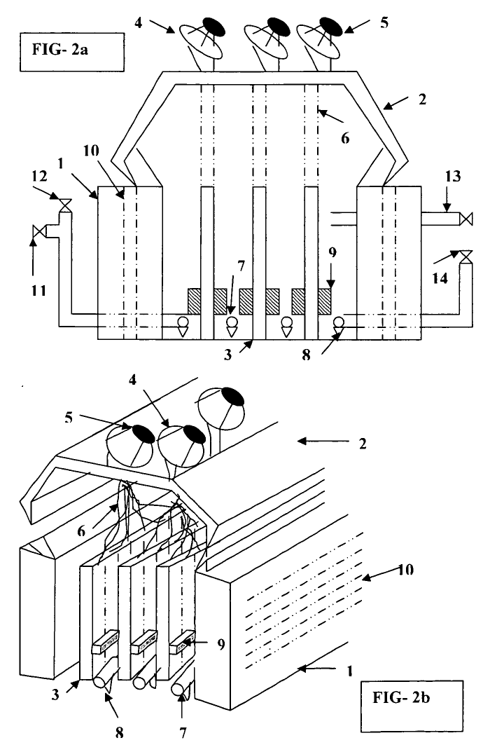

FIGS. 2a, b: Shows a cross-section side view of an alternate embodiment of the first preferred underground photobioreactor system, having plurality of circular hybrid solar collector system (4) and hybrid fiber-optics light guide (3) for autotrophic or heterotrophic cultivation of one or more photoautotrophic organism that require low level of light while using organic carbon as an energy source. In this exemplary configuration, each of the vertically arranged fiber-optic light guides could be circular or flat with variable thickness depending upon the needs of the organism and amount of dispersion of light needed to maintain the photoperiod and the dark and light cycle of the organism.

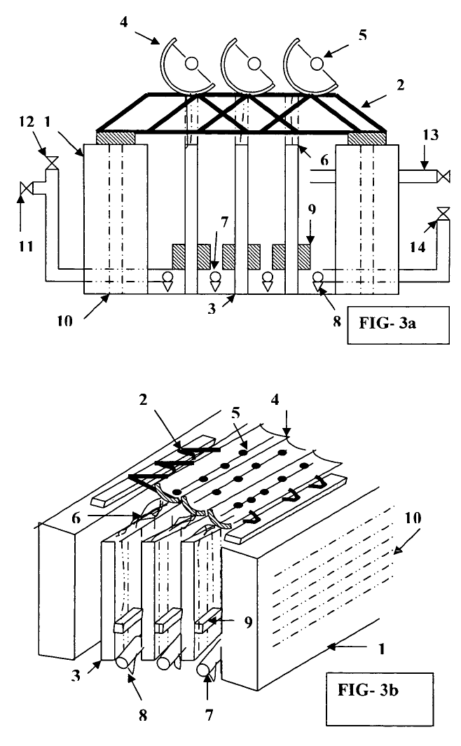

FIGS. 3a, b: Shows cross-section side view of an alternate embodiment of an open top underground algal raceway system with hybrid solar trough (4) attached to support structure (2). A flat hybrid optic light guide (3) is connected with fiber-optics and electro luminescence ropes (6) to disperse solar light to depth of 25 ft or more depending upon the light requirement of the algae. The design is suitable for large scale controlled cultivation for food and bio-diesel grade algae. The system is beneficial for photoautotrophic organisms that have extended light requirement, and is facilitated by electro luminescence ropes in night. Mixing in commercial system can be performed by paddle wheel, air lift, wave maker or other mechanical means to keep the photoautotrophic organism in suspension. These equipments are known technology and not shown for simplicity of illustration.

FIGS. 4a, b: Shows a close up image of fiber optic light guide and cleaning system. The fiber optic light guide consist of two sheets of casing (1) made of transparent smooth, heat, chemical and light resistant material not limited to glass, acrylic, polymer, quarts etc. Fiber optic or electro luminescence rope (8) are inserted within groves on the casing and sealed on the extremities by teflon gasket or gasket made by other non reactive non growth forming material so that the channel is water tight. The fiber optic light guide can be placed directly into the reactor or contained in a transparent housing (7) made of the similar material as used in light guide casing. A space between light guide and the housing is to be filled by a light filtering fluid for spectral control. Additional light filters can be placed in photo collectors, a close-up represented in FIGS. 5a, b, (3) to deliver specific wavelength needs of photoautotrophic organism.

Even though the channels are made of smooth non binding material but with time there are chances of the culture or media crystals to bind on it. If this happens, the efficiency of light transmission can reduce thereby reducing the photoautotrophic organism growth. To avoid this problem we have developed an automatic channel cleaning system that operates without shutting down the process for cleaning. The automatic channel cleaner is a low maintenance sliding or movable device comprising a plurality of cleaning devices adapted to clean the outer surface of light guide or its housing preferably but not limited to rake, or brush like structure or high pressure air nozzle moving on hydraulic telescopic cylinder, ball-nut and screw, rail or pulley system.

The cleaning system is mounted over the light guide (1) or housing (7). A cleaning adaptor is attached to a support frame (4). The support frame has a ball-nut assembly like connector similar to one represented in U.S. Pat. Nos. 4,211,125 and 6,089,117 (3) through which an endless screw actuator (2) is passed, a person skilled in art will understand that other mechanically moving equipment such as hydraulic telescopic cylinder rail or pulley system can replace the screw and connector system. The endless screw is rotated by gear (6) which is in turn, driven by a drive (not shown) for actuating the cleaning means preferably along the entire length of the light guide housing. The cleaning adaptor has plurality of bristles or rubber rake or air nozzles (5) on the inner side facing the light guide housing. If the adaptor is attached with air nozzle and additional air connection shall be hooked up that is not shown in the drawing for simplicity of illustration.

FIGS. 5a, b: Illustrates off the shelf hybrid solar collector similar to the one shown in U.S. Pat. No. 6,603,069, will be used in present invention to deliver full spectrum or selective wavelength of solar energy in accordance to the algae needs by efficiently converting concentrated cool light and utilizing the remaining infra red energy for electricity generation

A circular or linear trough primary mirror (8) concentrates light into secondary optical element (3) that is sectioned into twelve or more surface, each shaped to reflect visible light into large core optical fibers (6) mounted on concentric fiber mount assembly (1). The secondary optical element also has non imaging optic concentrator to uniformly distribute infrared radiation into Infrared-Thermo Photovoltaic Assembly. The secondary optical element is placed at a defined distance with help of guides (4) that is linked to a metallic plate or rare earth metal ring (2) and a secondary metallic plate or rare earth magnet on the other side (5).

In the linear solar trough there is a grove (7) that is used to adjust the number of secondary element on a trough and also according to the fiber optic guide placement. The large core fiber optics are placed in channel or guide or attached to diffusing rod to deliver even illumination for algae. Fiber configuration to optimize side-lighting efficiency of large core optical fiber are used, this design increases the surface area illuminated and drastically reduce photosynthetic saturation while demonstrating the ability to achieve much higher volumetric carbon fixation rates, filtering unwanted UV and infrared radiation that cause fluctuation in media temperature, and increasing the overall sunlight utilization efficiency.

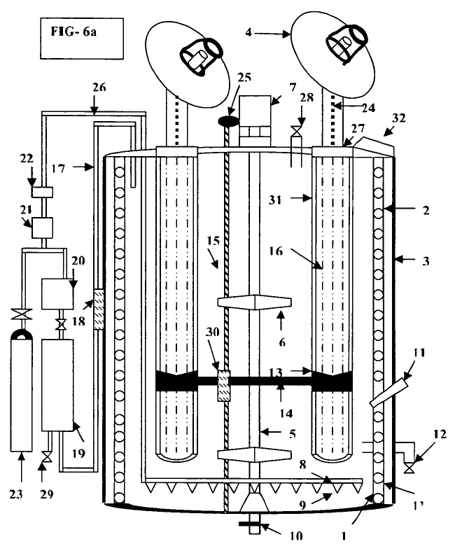

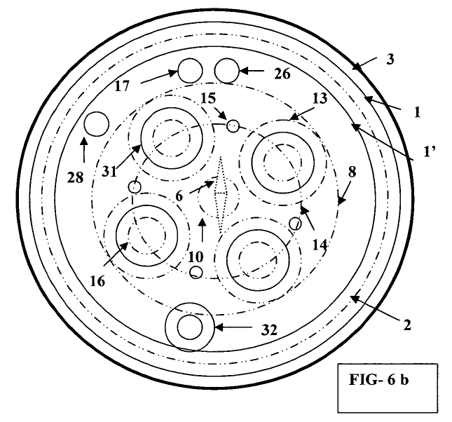

FIG. 6a: Shows a longitudinal cross section view of “above ground” sealed double wall (1, 1′) tank made of polymer, metal or other non-reactive non-corrosive material, covered with insulation (3). Between the inner tank and outer tank wall there is a heating or cooling coil (2) that contains control flow of circulating water to regulate the temperature of photo bioreactor system. Air inlet pipe (26) with safety valve (22) and compressor (21) supplies air to the coil shape or annular gas emitter pipe (8) attached with nozzles (9) directed obliquely downward. The venting line (17) is connected to carbon dioxide or Oxygen concentration detector (18) and enters a catalytic converter (19) into which hydrogen is introduced in a controlled manner (29) to react with molecular oxygen in the presence of catalyst. The water vapor formed by combustion reaction is collected into the condenser (20). The oxygen free air is mixed with controlled dose of carbon dioxide from the carbon dioxide tank (23). The photo-bioreactor has securely mounted (27) minimum one hybrid solar collector (4) and tubular hybrid optics guide (16) to fulfill the photosynthetic needs of the algae. Each light guide is placed into a transparent housing (31) and the space between the light guide and the housing shall be filled by light filtering fluid for spectral control. The photo-bioreactor further comprises at least one cleaning adaptor (13) that is mounted over the light guide housing (31) for cleaning the outer surface of the housing. The cleaning adaptor is attached to support frame (14) that has a ball-nut like adaptor (30) through which an endless screw actuator (15) is passed.

The unit shall be consisting an agitator shaft (5) and agitator vanes (6) driven by electric motor (7) to uniformly disperse gas into nutrient medium, and keep the algae suspended in close contact of the light source, in accordance to their light and dark cycle requirement.

The bioreactor comprises minimum one dosing port (28) and one product collection port (10, 12). The photo bioreactor shall also have a computer controlled system and probes to continuously monitor pH, DOT, Hydrogen, Nitrogen, light meter, pressure, temperature and inline biomass detector (11). The variable light intensity or flashing light shall be controlled to optimum level of cell suspension density.

FIG. 6b Shows a plan view of FIG. 6(a) embodiment showing four hybrid optic tubular guides (16) within a housing (31) placed equidistantly in the photo-bioreactor.

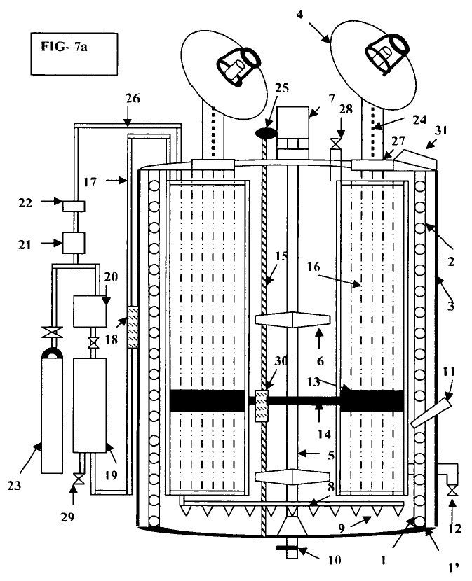

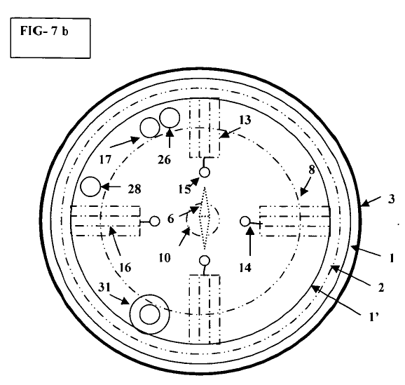

FIG. 7a: In an alternate embodiment of “above ground” sealed photo-bioreactor with flat hybrid light guides (16) consisting of fiber optic and electro luminescence are used instead of tubular light guides. Additionally the cleaning adaptor (13) is long flat instead of circular shaped.

FIG. 7b: Shows a plan view of FIG. 7(a) embodiment showing four hybrid optic flat guides (16) placed equidistantly within the photo-bioreactor.

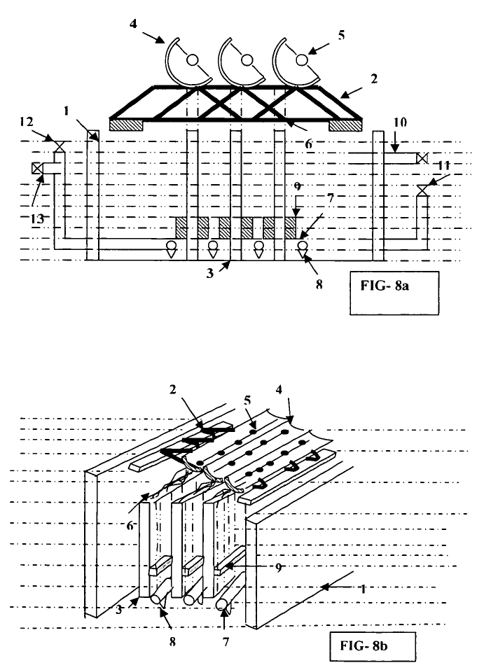

FIGS. 8a, b: Shows a cross sectional view of an open top floating photo-bioreactor system in ocean surface with hybrid solar collector in accord with the present invention. Utilization of floating Photobioreactor system is the most cost efficient way to grow photoautotrophic organism. The system has ample water to operate, can be moved to place with high solar radiation, does not compete with agricultural land, cheap to construct, can be used in river streams or bays where high nitrogen and phosphorus pollutions cause blooms.

The walls of the photobioreactor (1) are made of air filled polymer bag or any other high strength light weight material that is water resistant and can hold in air pressure. The system has plurality of floating solar trough (4) on air filled support (2) with multiple photo-collectors (5) linked to fiber optic strands (6) and fiber-optics light guides (3). The light guides can be as flat curtains or separators or circular with varying thickness depending upon the light needs of the photoautotrophic organism. Solar energy collection units are arranged on a sun facing surface that is exposed to a sufficient duration of sunlight for solar energy collection. In a preferred embodiment, the methodology of the present invention entails providing fluid supply channel (10) and/or fluid release channel (11) to the enclosed photobioreactor.

The biomass is harvested at the location and the clear fluid coming out of the fluid outlet is filtered and blended with nutrients before it is sent back to the photobioreactor through the fluid supply channel. In the exemplary, configuration, each of the horizontally arranged injector sparger gas contacting system (7) is placed facing nozzle down (8); and operated by use of pneumatic valves to control the oxygen, carbon dioxide ratio in the medium (12, 13). This system also helps the algae to be suspended and in motion.

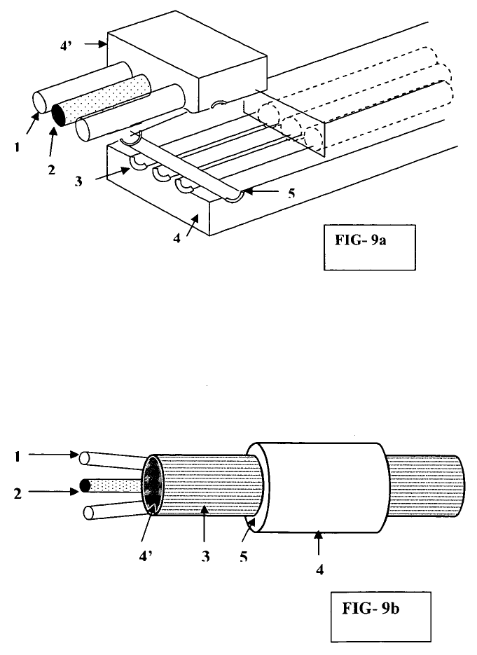

FIGS. 9a, b: Is a cross sectional view of an alternate embodiment of a tubular or flat hybrid fiber optic channel. The side lighting fiber optic strands (1) or electro luminescence rope (2) or a combination are placed within two transparent sheets (4, 4′). The material used for the hybrid light guide and housing shall be transparent, UV stable, water resistant, non reactive, smooth surface with or without diffusers. The double layer sheet is sealed by placing a teflon gasket or gasket made by other non reactive non growth forming material into the grove (5) so that the channel is water tight. In addition only two fiber optic strand and one electro luminescence rope is shown per light guide for simplicity of illustration. It should be understood that the quantity of fiber optic and electro luminescence rope and size or diameter of light guide can be scaled to accommodate the needs of optimum productivity of the desired photoautotrophic organism.

### EXAMPLE

The function and advantage of the present invention may be more fully understood from the examples below.

The challenge test was performed on Chlorella to analyze their growth kinetics, culture stability, effect of light intensity and parameter influences. In a pilot-scale photobioreactor system, the algae were exposed to a set of different medium and controlled environmental condition that are specifically selected to simulate operative conditions to which the algae will be exposed in the photobioreactor such as liquid medium composition; temperature; pH fluctuation; light intensity variation; light and dark exposure durations and light/dark transition frequency and pattern.

The following examples, is illustrative of certain embodiments of the invention, and do not exemplify the full scope of the invention.

A 700 L close top photobioreactor as represented in FIG. 1 with a working volume of 600 L was inoculated with Chlorella pyrenoidosa 0.01 g/l into modified Knop's medium with the following components: 0.5 g KNO3, 0.5 g Ca(NO3)2.4H2O, 0.2 g KH2PO4, 0.15 g MgSO4.7H2O, 0.01 g FeCl3.6H2O, 0.003 g H3BO3, 0.002 g MnCl2.4H2O, 0.0003 g NH4VO3, 0.0002 g ZnSO4.7H2O, 0.0001 g (NH4)6Mo7O24.7H2O in 1 L of distilled water. Working parameters were temperature of 25° C.±1° C., pH 6.8 and 16 h photoperiod. The process was run continuously for 120 days under sterile conditions and generated the maximum biomass densities of 3.6 g/L day with a composition of 59.5% protein, 22.8% lipid and 9.2% carbohydrate.

After 120 days of autotrophic cultivation the process was changed to heterotrophic cultivation for additional 30 days by dosing corn powder hydrolysate as organic carbon, giving a significant improvement in cell density of 5.4 g/L in 72 hrs with a chemical composition of 11.1% protein, 56.2% lipid and 14.1% carbohydrate.

### References Cited

### U.S. PATENT DOCUMENTS

| | | |
|---|---|---|
| 5,137,828 | 11 Aug. 1992 | Robinson et al. |
| 5,242 827 | 07 Sep. 1993 | Chaumont et al. |
| 6,174,720 | 16 Jan. 2001 | Oxley et al. |
| 5,104,803 | 14 Apr. 1992 | Delente |
| 5,162,051 | 10 Nov. 1992 | Hoeksema |
| 5,614,378 | 25 Mar. 1997 | Yang et al. |
| 6,603,069 | 05 Aug. 2003 | Muhs et al. |
| 4,211,125 | 08 Jul. 1980 | Benton |
| 6,089,117 | 18 Jul. 2000 | Ebina et al. |

### Other Publications

Ogbanna, J. C. and Tanaka, H. (1997), Industrial-size photobioreactors. Chemtech: 27(7) 43-49.

What is claimed is:

1. A close top photobioreactor for mass cultivation of photoautotrophic organism in liquid pool, comprising a core support structure which is upstanding and substantially raceway shape with a liquid inlet and outlet port.

2. The photobioreactor according to claim 1 further comprises an air inlet port that is linked to the injector sparger gas system that provides a controlled carbon dioxide delivery.

3. The photobioreactor according to claim 1 further comprises a plurality of fiber optic light guide or hybrid light emitting guides in tubular or flat shape that are immersed into the growth media, and mounted within the container extending in a substantially parallel way from the said first wall to said second wall such that the light source can illuminate the liquid pool uniformly.

4. This light guide as mentioned in claim 3 can be installed in photobioreactor directly or within a housing and the space in-between the light guide and the housing is filled with a light filtering fluid for spectral control.

5. Said hybrid light emitting guide in claim 3 are connected to at least one hybrid solar collector installed on a circular or trough shaped primary mirror to deliver full spectrum or selective wavelength of solar energy in accordance to the algae needs by efficiently concentrating cool light and utilizing the remaining infrared energy for electricity generation.

6. The photobioreactor according to claim 1 further comprises an automatic hybrid light emitting guide cleaning adaptor that is installed over the light guides or housing and operates without shutting down the process for cleaning the buildup on the light guides that could result in reducing the efficiency of light transmission.

7. The photobioreactor according to claim 1 further comprises a sliding or moving device similar to endless screw actuator and ball-nut assembly that is rotated by gear and drive to move the cleaning adaptor preferably along the entire length of the light guide or housing.

8. The photobioreactor according to claim 1 further comprises of a geothermal heat exchange tubes made of smooth heat transferring material, within the support structure wall to control the photobioreactor temperature.

9. The photobioreactor according to claim 1 further comprises at least one fluid moving equipment such as paddle wheel, stirrer, wave maker to keep the photoautotrophic organism suspended near the light source.

10. A cleaning adaptor according to claim 6 where the inner side comprises a cleaning means such as brush, rake or high pressure air nozzle for cleaning the outer surface of the light guide or its housing.

11. The photobioreactor according to claim 1 further comprises a lid for hermetically enclose the liquid pool in said vessel of said photobioreactor. Over the lid at least one hybrid solar collector as in claim 5 is installed and access ends for the fiber optic strands and electro luminescence are provided with protective cover.

12. An alternate embodiment of claim 1 where the photobioreactor is an open top mass cultivation system and has the injector sparger gas system according to claim 2, light guide according to claim 3, light guide housing according to claim 4 hybrid solar collector according to claim 5, guide cleaning adaptor according to claims 6 and 10, moving device for cleaning adapter according to claim 7, geothermal heating exchange tubes according to claim 8, fluid moving equipment according to claim 9.

13. An alternate embodiment of claim 12 where the open top floating photo-bioreactor system is on ocean surface. The walls of the photobioreactor are made of air filled polymer bag or any other high strength light weight material that is water resistant and can hold in air pressure. The system has plurality of floating solar trough floating on air filled support.

14. A second preferred embodiment of an above ground sealed photobioreactor assembly preferably cylindrical shape, having outer and inner tank, the outer tank is covered with insulation. In-between the two tanks there is a heating or cooling coil as a means of temperature control.

15. An inner tank according to claim 14, to accommodated fluid media is being provided with (i) one dosing port and one product collection port (ii) one air pipe with safety valve and compressor that supplies air to the annular gas emitter pipe which is circular or coil shape with nozzles directed obliquely downward. (iii) agitator shaft and agitator vanes driven by electric motor to uniformly disperse gas into nutrient medium, and keep the algae suspended in close contact of the light source, (iv) computer controlled system and probes to continuously monitor pH, DOT, hydrogen, nitrogen, light meter, pressure, temperature and inline biomass detector.

16. A photobioreactor tank assembly according to the claim 14 has securely mounted minimum one hybrid solar collector and minimum one optically transparent fiber optic or hybrid optic light guide.

17. Each light guide according to claim 16 is placed directly into the tank or placed into a transparent housing and the space between the light guide and the housing shall be filled by light filtering fluid for spectral control.

18. The photo-bioreactor according to claim 14 further comprises at least one cleaning adaptor according to claims 6, 7 and 10 for cleaning the outer surface of the housing.

19. The photo-bioreactor according to claim 14 further comprises close loop venting line connected to Carbon dioxide or Oxygen concentration detector and a catalytic converter into which hydrogen is introduced in a controlled manner to react with molecular oxygen in the presence of catalyst. The water vapor formed by combustion reaction is collected into the condenser and the oxygen free air is mixed with controlled dose of Carbon dioxide from the carbon dioxide tank before it enters the reactor again.

20. The photobioreactor according to claim 1, claim 12, claim 13, claim 14 can have continues delivery of full spectrum or selective wavelength of solar energy or delivered as flashing light in accordance to their light and dark cycle requirement of the photoautotrophic organism or suspension density. For organism needing extended light requirement beyond the daylight period, electro luminescence shall be an economical source of light.

21. The photobioreactor according to claim 1, claim 12, claim 13, claim 14 consist of off the shelf hybrid solar collectors to deliver full spectrum or selective wavelength of infrared filtered cool solar light.
